(19) United States
(12) Patent Application Publication
(10) Pub. No.: US 2026/0354738 A1
(43) Pub. Date: Nov. 12, 2026

(54) METHOD TO QUANTIFY AUTONOMIC NERVOUS SYSTEM ACTIVITY AND TO IDENTIFY POTENTIAL RESPONDERS TO AUTONOMIC MODULATION THERAPY

(71) Applicant: Medtronic Ireland Manufacturing Unlimited Company, Dublin 2 (IE)

(72) Inventors: Douglas A. Hettrick, Andover, MN (US); Sean Michael White, Rancho Santa Margarita, CA (US); Darion R. Peterson, Longmont, CO (US); Paul J. Coates, Corte Madera, CA (US)

(73) Assignee: Medtronic Ireland Manufacturing Unlimited Company, Dublin 2 (IE)

(21) Appl. No.: 19/132,666

(22) PCT Filed: Nov. 16, 2023
(86) PCT No.: PCT/EP2023/082088

§ 371 Date: May 23, 2025

Related U.S. Application Data
(60) Provisional application No. 63/427,574, filed on Nov. 23, 2022.

(51) Int. Cl. A61B 5/00; A61B 5/021; A61B 5/026

(52) U.S. Cl. CPC A61B 5/4035; A61B 5/0002; A61B 5/021; A61B 5/0261; A61B 5/6801; A61B 5/7257; A61B 5/7278

(57) ABSTRACT

A system for performing a diagnostic procedure includes a diagnostic device configured for placement proximate tissue having a light emitter and a photodetector, a computing device which delivers light from the light emitter into the tissue for a period of time, detects dynamic properties of light scattering particles within the tissue using the photodetector, generates a peripheral blood flow waveform of a fluid flowing within the tissue using the detected dynamic properties, calculates a power spectral density for the generated peripheral blood flow waveform, quantifies a relative level of global sympathetic nerve activity using the calculated power spectral density, identifies relative changes in global sympathetic nerve activity within the quantified relative level of global sympathetic nerve activity, and determines if the identified relative changes in global sympathetic nerve activity fall outside of a predetermined range of global sympathetic nerve activity, which is indicative of a candidate for therapy.

This application claims the benefit of U.S. Provisional Patent Application Ser. No. 63/427,574, filed Nov. 23, 2022, the entire content of which is incorporated herein by reference.

### TECHNICAL FIELD

The present disclosure relates generally to quantifying sympathetic nerve activity. In particular, the disclosure is directed to diagnostic methods and systems for quantifying sympathetic nerve activity or changes in sympathetic nerve activity to identify diseases associated with autonomic nervous system function.

### BACKGROUND

The sympathetic branch, or sympathetic nervous system (SNS), of the autonomic nervous system is critical to controlling multiple organs and physiologic systems, including the kidney. The SNS is a primarily involuntary bodily control system typically associated with stress response. Chronic over-activation of the SNS is a maladaptive response that can drive the progression of many disease states. For example, excessive activation of the renal SNS has been identified experimentally and in humans as a likely contributor to the complex pathophysiology of arrhythmias, hypertension, states of volume overload (e.g., heart failure), obstructive sleep apnea, and progressive renal disease, amongst others. Quantifying the relative level of global sympathetic activation within the human body, or relative changes in sympathetic activity due to various stimuli have multiple useful clinical applications, including diagnosing and monitoring diseases associated with autonomic nervous system function.

### SUMMARY

In accordance with the present disclosure, a system for performing a diagnostic procedure includes a diagnostic device, the diagnostic device configured for placement proximate tissue, wherein a light emitter and a photodetector are disposed on the diagnostic device, a computing device including a processor and a memory storing instructions, which when executed by the processor, cause the computing device to deliver light from the light emitter into the tissue for a period of time, detect dynamic properties of light scattering particles within the tissue using the photodetector over the period of time, generate a peripheral blood flow waveform of a fluid flowing within the tissue using the detected dynamic properties of the light scattering particles over the period of time, calculate a power spectral density for the generated peripheral blood flow waveform over the period of time, quantify a relative level of global sympathetic nerve activity over the period of time using the calculated power spectral density for the generated peripheral blood flow waveform, identify relative changes in global sympathetic nerve activity within the quantified relative level of global sympathetic nerve activity over the period of time, and determine if the identified relative changes in global sympathetic nerve activity fall outside of a predetermined range of global sympathetic nerve activity, wherein one or more identified relative changes in global sympathetic nerve activity falling outside of the predetermined range is indicative of a candidate for therapy.

In aspects, the instructions, when executed by the processor, may cause the computing device to calculate the power spectral density for the generated peripheral blood flow waveform over the period of time using a Fourier transform.

In certain aspects, calculating the power spectral density may include calculating the power spectral density for the generated peripheral blood flow waveform over the period of time using the fast Fourier transform.

In other aspects, the instructions, when executed by the processor, may cause the computing device to calculate an aortic input impedance spectrum for the generated peripheral blood flow waveform over the period of time using the calculated power spectral density for the generated peripheral blood flow waveform.

In certain aspects, the instructions, when executed by the processor, may cause the computing device to quantify the relative level of global sympathetic nerve activity over the period of time using one of the calculated power spectral density of the calculated aortic impedance spectrum.

In other aspects, the instructions, when executed by the processor, may cause the computing device to calculate the aortic input impedance spectrum for the generated peripheral blood flow waveform over the period of time using a Windkessel model.

In aspects, the instructions, when executed by the processor, may cause the computing device to calculate the aortic input impedance spectrum for the generated peripheral blood flow waveform over the period of time using the three-element Windkessel model.

In other aspects, the instructions, when executed by the processor, may cause the computing device to calculate an amount of light received by the photodetector over the period of time.

In certain aspects, the instructions, when executed by the processor, may cause the computing device to generate an arterial blood pressure waveform of the fluid flowing within the target tissue using the calculated amount of light received by the photodetector over the period of time.

In aspects, the instructions, when executed by the processor, may cause the computing device to calculate the aortic input impedance spectrum of a combined generated peripheral blood flow waveform and generated arterial blood pressure waveform.

In certain aspects, the instructions, when executed by the processor, may cause the computing device to combine the generated peripheral blood flow waveform and the generated arterial blood pressure waveform using a derived one or more of a determined resistance, compliance, amplitude, or phase from the generated peripheral blood flow waveform and the generated arterial blood pressure waveform.

In other aspects, the instructions, when executed by the processor, may cause the computing device to calculate a power spectral density of a combined generated peripheral blood flow waveform and generated arterial blood pressure waveform.

In certain aspects, the instructions, when executed by the processor, may cause the computing device to combine the generated peripheral blood flow waveform and the generated arterial blood pressure waveform using a derived mean arterial blood pressure amplitude and a derived mean peripheral blood flow waveform from the generated peripheral blood flow waveform and the generated arterial blood pressure waveform over the period of time.

In aspects, the method may include intentionally applying a sympathetic stimulus to the candidate for therapy, wherein identifying relative changes in global sympathetic nerve activity includes identifying relative changes in global sympathetic nerve activity during the intentionally applied sympathetic stimulus.

In other aspects, the intentionally applied sympathetic stimulus may be selected from the group consisting of a cold pressor, a hand rip exercise, mental stress, a Valsalva or Mueller maneuver, an acute catécholamine injection, orthostasis, and variable rate pacing.

In certain aspects, the instructions, when executed by the processor, may cause the computing device to identify relative changes in global sympathetic nerve activity due to naturally occurring sympathetic stimulus.

In aspects, the naturally occurring sympathetic stimulus may be selected from the group consisting of circadian variation, sleep apnea breathing, orthostasis, intense physical activity, and respiration.

In certain aspects, the diagnostic device may be a portable, wearable device, the portable, wearable device wirelessly couplable to the computing device.

In other aspects, the diagnostic device may be operably coupled to the computing device using a wired connection.

In aspects, the instructions, when executed by the processor, may cause the computing device to issue one or more of a clinical action, an alert, or a recommendation based upon whether the identified relative changes in global sympathetic nerve activity fall outside of the predetermined range.

In accordance with another aspect of the present disclosure, a method of assessing a candidate for therapy includes emitting light, by a computing device, into target tissue from a light emitter over a period of time, receiving, by the computing device, from a photodetector, a signal indicative of dynamic properties of light scattering particles within the target tissue over the period of time, generating, by the computing device, a peripheral blood flow waveform of a fluid flowing within the target tissue using the detected dynamic properties of the light scattering particles over the period of time, calculating, by the computing device, a power spectral density for the generated peripheral blood flow waveform over the period of time, quantifying, by the computing device, a relative level of global sympathetic nerve activity over the period of time using the calculated power spectral density for the generated peripheral blood flow waveform, identifying, by the computing device, relative changes in global sympathetic nerve activity within the quantified relative level of global sympathetic nerve activity over the period of time, and determining, by the computing device, if the identified relative changes in global sympathetic nerve activity fall outside of a predetermined range of global sympathetic nerve activity, wherein one or more identified relative changes in global sympathetic nerve activity falling outside of the predetermined range is indicative of a candidate for therapy.

In aspects, calculating the power spectral density may include calculating the power spectral density for the generated peripheral blood flow waveform over the period of time using a Fourier transform.

In other aspects, calculating the power spectral density may include calculating the power spectral density for the generated peripheral blood flow waveform over the period of time using the fast Fourier transform.

In certain aspects, the method may include calculating, by the computing device, an aortic input impedance spectrum for the generated peripheral blood flow waveform over the period of time using the calculated power spectral density for the generated peripheral blood flow waveform.

In other aspects, quantifying the relative level of global sympathetic nerve activity may include quantifying the relative level of global sympathetic nerve activity over the period of time using one of the calculated power spectral density or the calculated aortic impedance spectrum.

In certain aspects, calculating the aortic input impedance spectrum may include calculating the aortic input impedance spectrum for the generated peripheral blood flow waveform over the period of time using a Windkessel model.

In other aspects, calculating the aortic input impedance spectrum may include calculating the aortic input impedance spectrum for the generated peripheral blood flow waveform over the period of time using the three-element Windkessel model.

In aspects, the method may include calculating, by the computing device, an amount of light received by the photodetector over the period of time.

In certain aspects, the method may include generating, by the computing device, an arterial blood pressure waveform of the fluid flowing within the target tissue using the calculated amount of light received by the photodetector over the period of time.

In aspects, calculating the aortic input impedance spectrum may include calculating the aortic input impedance spectrum of a combined generated peripheral blood flow waveform and generated arterial blood pressure waveform.

In other aspects, combining the generated peripheral blood flow waveform and the generated arterial blood pressure waveform may include deriving one or more of a determined resistance, compliance, amplitude, or phase from the generated peripheral blood flow waveform and the generated arterial blood pressure waveform.

In certain aspects, calculating the power spectral density may include calculating a power spectral density of a combined generated peripheral blood flow waveform and generated arterial blood pressure waveform.

In other aspects, combining the generated peripheral blood flow waveform and the generated arterial blood pressure waveform may include deriving a mean arterial blood pressure amplitude and a mean peripheral blood flow waveform amplitude from the generated peripheral blood flow waveform and the generated arterial blood pressure waveform over the period of time.

In aspects, the method may include intentionally applying a sympathetic stimulus to the candidate for therapy, wherein identifying relative changes in the global sympathetic nerve activity includes identifying relative changes in global sympathetic nerve activity during the intentionally applied sympathetic stimulus.

In other aspects, the intentionally applied sympathetic stimulus may be selected from the group consisting of a cold pressor, a hand rip exercise, mental stress, a Valsalva or Mueller maneuver, an acute catécholamine injection, orthostasis, and variable rate pacing.

In certain aspects, identifying relative changes in global sympathetic nerve activity may include identifying relative changes in global sympathetic nerve activity due to naturally occurring sympathetic stimulus.

In aspects, the naturally occurring sympathetic stimulus may be selected from the group consisting of circadian variation, sleep apnea breathing, orthostasis, intense physical activity, and respiration.

In certain aspects, the method may include issuing one or more of a clinical action, an alert, or a recommendation based upon whether the identified relative changes in global sympathetic nerve activity fall outside of the predetermined range.

In accordance with another aspect of the present disclosure, a method of assessing and performing a therapeutic procedure includes navigating a therapeutic device to target tissue, the therapeutic device configured to apply therapy to the target tissue, applying therapy, by the therapeutic device, to the target tissue, during the application of therapy to the target tissue, monitoring, by a computing device, global sympathetic nerve activity, wherein monitoring global sympathetic nerve activity includes emitting light, from a light emitter, into tissue over a period of time, detecting, by the computing device, dynamic properties of light scattering particles within the tissue by a photodetector over the period of time, generating, by the computing device, a peripheral blood flow waveform of a fluid flowing within the tissue using the detected dynamic properties of the light scattering particles over the period of time, calculating, by the computing device, a power spectral density for the generated peripheral blood flow waveform over the period of time, quantifying, by the computing device, a relative level of global sympathetic nerve activity using the calculated power spectral density for the generated peripheral blood flow waveform, identifying, by the computing device, relative changes in global sympathetic nerve activity within the quantified relative level of global sympathetic nerve activity over the period of time, and determining, by the computing device, if the identified relative changes in global sympathetic nerve activity falls outside of a predetermined range of global sympathetic nerve activity, and terminating the application of therapy if the determined relative changes in global sympathetic nerve activity fall outside of the predetermined range.

In aspects, the method may include adjusting, by the computing device, the applied therapy in response to the monitored global sympathetic nerve activity.

In other aspects, the method may include, after the application of therapy to the target tissue, monitoring, by the computing device, global sympathetic nerve activity a second time to determine, by the computing device, an efficacy of the application of therapy to the target tissue.

In certain aspects, calculating a power spectral density may include calculating the power spectral density for the generated peripheral blood flow waveform over the period of time using a Fourier transform.

In aspects, calculating the power spectral density may include calculating the power spectral density for the generated peripheral blood flow waveform over the period of time using the fast Fourier transform.

In other aspects, the method may include calculating, by the computing device, an aortic input impedance spectrum for the generated peripheral blood flow waveform over the period of time using the calculated power spectral density for the generated peripheral blood flow waveform.

In aspects, quantifying the relative level of global sympathetic nerve activity may include quantifying the relative level of global sympathetic nerve activity over the period of time using one or the calculated power spectral density or the calculated aortic input impedance spectrum.

In certain aspects, calculating the aortic input impedance spectrum may include calculating the aortic input impedance spectrum for the generated peripheral blood flow waveform over the period of time using a Windkessel model.

In other aspects, calculating the aortic input impedance spectrum may include calculating the aortic input impedance spectrum for the generated peripheral blood flow waveform over the period of time using the three-element Windkessel model.

In aspects, the method may include calculating, by the computing device, an amount of light received by the photodetector over the period of time.

In certain aspects, the method may include generating, by the computing device, an arterial blood pressure waveform of the fluid flowing within the target tissue using the calculated amount of light received by the photodetector over the period of time.

In other aspects, calculating the aortic input impedance spectrum may include calculating the aortic input impedance spectrum of a combined generated peripheral blood flow waveform and generated arterial blood pressure waveform.

In certain aspects, combining the generated peripheral blood flow waveform and the generated arterial blood pressure waveform may include deriving one or more of a determined resistance, compliance, amplitude, or phase from the generated peripheral blood flow waveform and the generated arterial blood pressure waveform.

In aspects, calculating the power spectral density may include calculating a power spectral density of a combined generated peripheral blood flow waveform and generated arterial blood pressure waveform.

In other aspects, combining the generated peripheral blood flow waveform and the generated arterial blood pressure waveform may include deriving a mean arterial blood pressure amplitude and a mean peripheral blood flow waveform from the generated peripheral blood flow waveform and the generated arterial blood pressure waveform over the period of time.

In certain aspects, emitting light into tissue from a light emitted and detecting dynamic properties of light scattering particles within the tissue by a photodetector may include the light emitter and the photodetector being disposed on a diagnostic device.

In aspects, the diagnostic device may be a portable, wearable device.

In other aspects, the diagnostic device may be disposed on a digit of a patient.

Further disclosed herein is a system for performing a diagnostic procedure that includes a diagnostic device configured for placement proximate tissue having a light emitter and a photodetector, a computing device which delivers light from the light emitter into the tissue for a period of time, detects dynamic properties of light scattering particles within the tissue using the photodetector, generates a peripheral blood flow waveform of a fluid flowing within the tissue using the detected dynamic properties, calculates a power spectral density for the generated peripheral blood flow waveform, quantifies a relative level of global sympathetic nerve activity using the calculated power spectral density, identifies relative changes in global sympathetic nerve activity within the quantified relative level of global sympathetic nerve activity, and determines if the identified relative changes in global sympathetic nerve activity fall outside of a predetermined range of global sympathetic nerve activity, which is indicative of a candidate for therapy.

### BRIEF DESCRIPTION OF THE DRAWINGS

Various aspects and embodiments of the disclosure are described hereinbelow with references to the drawings, wherein:

FIG. 1 is a schematic diagram of a system provided in accordance with the present disclosure;

FIG. 2 is a schematic view of a workstation of the system of FIG. 1;

FIG. 3 is a perspective view of a therapeutic device of the system of FIG. 1;

FIG. 4 is a perspective view of a diagnostic device of the system of FIG. 1 shown disposed on a portion of a patient's anatomy;

FIG. 5 is a graphical representation of a user interface of the system of FIG. 1 illustrating a flow waveform;

FIG. 6 is a graphical representation of the user interface of FIG. 5 illustrating photoplethysmograpic (PPG) signals;

FIG. 7A is a pre-processing graphical representation of the flow waveform and the PPG signal of FIGS. 5 and 6;

FIG. 7B is a graphical representation of a Power Spectral Density (PSD) calculated for each of the flow waveform and the PPG signal of FIG. 7A;

FIG. 7C is a graphical representation of a power spectral analysis of arterial mechanical properties using an aortic input impedance spectrum;

FIG. 8A is a schematic representation of an algorithm for performing the power spectral analysis of FIG. 7C;

FIG. 8B is another schematic representation of an algorithm for performing the power spectral analysis of FIG. 7C;

FIG. 9 is a flow chart illustrating a method of performing a diagnostic procedure in accordance with the present disclosure;

FIG. 10 is a flow chart illustrating another embodiment of a method of performing a diagnostic procedure in accordance with the present disclosure; and

FIG. 11 is a flow chart illustrating a method of assessing and performing a therapeutic procedure provided in accordance with the present disclosure.

### DETAILED DESCRIPTION

This disclosure is directed to diagnostic systems and methods for quantifying sympathetic nerve activity or changes in sympathetic nerve activity to identify diseases associated with autonomic nervous system function. As can be appreciated, the diagnostic systems and methods described herein may be utilized to assess a candidacy of patients for renal denervation (RDN) therapy or other similar therapies for the treatment of diseases caused by over-activation of the SNS. The following description focuses on navigation to, an assessment of, and an application of therapy to the renal artery to identify candidate tissue and denervate sympathetic or, in certain embodiments, parasympathetic, nerves in, around, and proximate the renal arteries. However, the present disclosure is not so limited and can be employed for denervating nerves accessible via any blood vessel described herein or other luminal tissue (e.g., a bile duct). Additionally, in some examples, the devices, systems, and techniques described herein may be used in denervating nerves at a plurality of target locations within a patient, e.g., at two target locations accessible via different blood vessels. As one example, the devices, systems, and techniques described herein may be used in denervating nerves that innervate the liver via an artery feeding the liver and denervating nerves that innervate a kidney via one or more renal arteries or renal vessels.

The diagnostic system utilizes laser speckle imaging to non-invasively quantify peripheral blood flow and utilizes photoplethysmograpic (PPG) to quantify changes in blood volume and an accompanying waveform. Such signals may be combined using models relating flow, volume, and pressure to non-invasively generate an arterial blood pressure waveform over a period of time. The system calculates Power Spectral Density (PSD) for both the peripheral blood flow measurements and the arterial blood pressure waveform over the period of time. PSD is a measure of a signal's power content versus frequency and may be calculated in the frequency domain using the Fourier transform (FT), which in embodiments, may be the fast Fourier transform (FFT).

The diagnostic system conducts a power spectral analysis of arterial mechanical properties using an aortic input impedance spectrum. The aortic input impedance spectrum quantifies frequency independent and dependent components of left ventricular afterload, such as peripheral arterial resistance and arterial compliance. The aortic input impedance spectrum is calculated using the quantified arterial blood flow measurements and the arterial blood pressure waveform. As can be appreciated, the arterial mechanical properties, such as peripheral arteriolar dimensions (e.g., resistance), aortic wall stiffness (e.g., compliance), wave reflections (e.g., reflectance), can be derived from the magnitude or phase of the aortic input impedance spectrum using a mechanical or electrical model of the arterial circulation. In one non-limiting embodiment, the aortic input impedance spectrum may be interpreted using a Windkessel model, such as the three-element Windkessel model.

The diagnostic system quantifies a relative level of global sympathetic nerve activity, as well as relative changes in activity, by analyzing components of the measured peripheral blood flow and PPG signals alone, or in combination, in the time or frequency domain. In this manner, the measured peripheral blood flow and the PPG signals can be analyzed in various combinations, such as individually in the time domain, individually in the frequency domain, combined in the time domain (e.g., the mean PPG signal amplitude and the mean measured peripheral blood flow amplitude), or combined in the frequency domain (e.g., resistance, compliance, amplitude, phase, etc.), amongst others. If analyzed in combination, it is envisioned that within each combination, various features of the signal can be derived, including both time domain (e.g., mean, maximum, minimum, etc.) or frequency domain parameters (e.g., magnitude, phase, etc.). Within the frequency domain, it is contemplated that the analysis may consist of low and high frequency bands, multiple bands, or any other frequency band of interest. In one non-limiting embodiment, the very low frequency band is analyzed, which is within the range of SNS firing (e.g., vasomotion).

Within each combination of the measured peripheral blood flow and the PPG signals, the diagnostic system can derive parameters indicative of SNS activity in the basal steady state or following a perturbation of the SNS. Perturbations of the SNS can be intentionally applied or could naturally occur in the case of long-term monitoring (e.g., over a single night, over several days and nights, etc.) of peripheral blood flow and PPG signals. Intentionally applied perturbations of the SNS may include a cold pressor, a hand rip exercise, mental stress (mental math, a stroop color test, etc.), a valsava or Müller maneuver, acute catecholamine injection (e.g., norepinephrine), orthostasis (e.g., rise from supine to standing), variable rate pacing (e.g., adjust the heat rate), amongst others. Naturally occurring perturbations of the SNS may include circadian variation (e.g., morning or evening surge), sleep apnea breathing, orthostasis (e.g., a rise from supine to standing that is detectable with a 3-D accelerometer), intense physical activity (e.g., a heart rate or activity increase), respiration, amongst others.

For each of the tracked peripheral blood flow and the PPG signal, a normal range or normal pattern of variation may be defined as well as one or more thresholds for detecting a deviation from the defined normal range or pattern of variation. As can be appreciated, the defined normal range or normal pattern of variation may be generated from data obtained from the patient or from clinical data obtained from an electronic medical records (EMR) system. It is envisioned that the defined normal range or normal pattern of variation may be expressed as either an absolute value or a proportional change without departing from the scope of the present disclosure. In embodiments, the measurements described herein may be made acutely (e.g., over a short period of time) or chronically (e.g., over a long period of time) using the same signals obtained from a similar portable or wearable device.

In embodiments, the diagnostic system may be utilized to assess the efficacy of denervation therapy applied to target tissue. In this manner, after the application of denervation therapy to the target tissue, the diagnostic device may once again provide parameters indicative of SNS activity in the basal steady state or following a perturbation of the SNS and provide an assessment of the efficacy of the applied denervation therapy. If the tracked peripheral blood flow or PPG signals deviate from the defined normal range or pattern of variation or otherwise exceed/fall below the predetermined thresholds, the application of denervation therapy has not been successful and further applications of denervation therapy may be needed. In contrast, if the tracked peripheral blood flow or PPG signals do not deviate from the defined normal range or pattern of variation or otherwise exceed/fall below the predetermined thresholds, the application of denervation therapy has been successful and no further applications of denervation therapy are needed.

It is envisioned that the diagnostic system device may be utilized concurrently with the application of denervation therapy to the target tissue. In this manner, during the application of denervation therapy, the diagnostic device tracks the peripheral blood flow or PPG signals during the application of denervation therapy and when the tracked peripheral blood flow or PPG signals no longer deviate from the defined normal range or pattern of variation or otherwise exceed/fall below the predetermined thresholds, the application of denervation therapy has been successful and the application of denervation therapy can be terminated. It is envisioned that the therapy system may modulate or otherwise change the intensity or duration of the application of denervation therapy based upon the tracked peripheral blood flow or PPG signals.

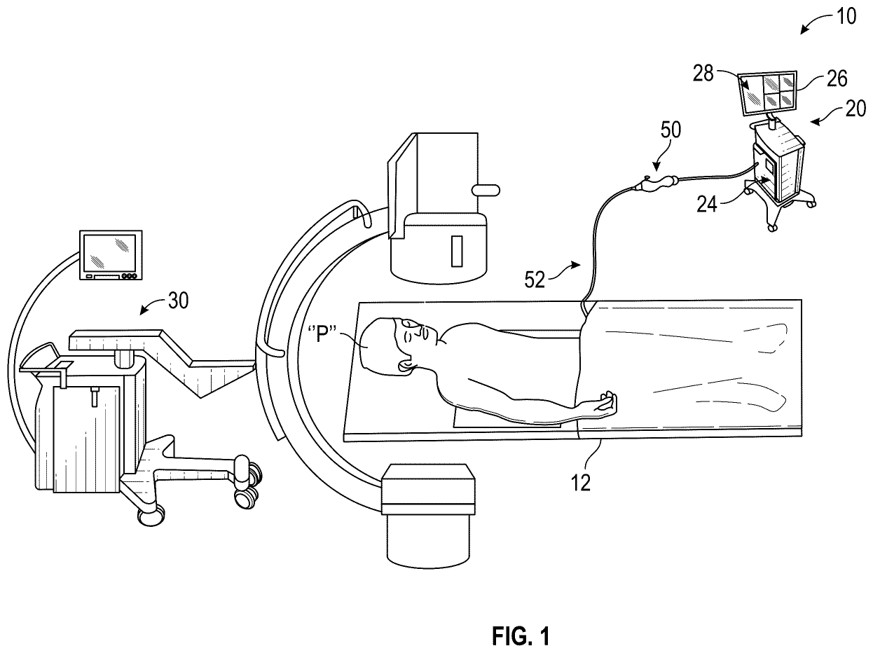

Turning now to the drawings, the diagnostic systems and methods described herein may be utilized to assess a candidacy of patients for renal denervation (RDN) therapy or other similar therapies for the treatment of diseases caused by over-activation of the SNS. FIG. 1 illustrates a guidance and therapy system provided in accordance with the disclosure and generally identified by reference numeral 10. As will be described in further detail hereinbelow, the guidance and therapy system 10 enables navigation of a therapeutic device 50 to a desired location within the patient's anatomy (e.g., the patient's renal artery), assessment of tissue within the renal artery for candidacy for denervation, and the application of denervation therapy to the tissue within the renal artery to denervate sympathetic nerves within the tissue.

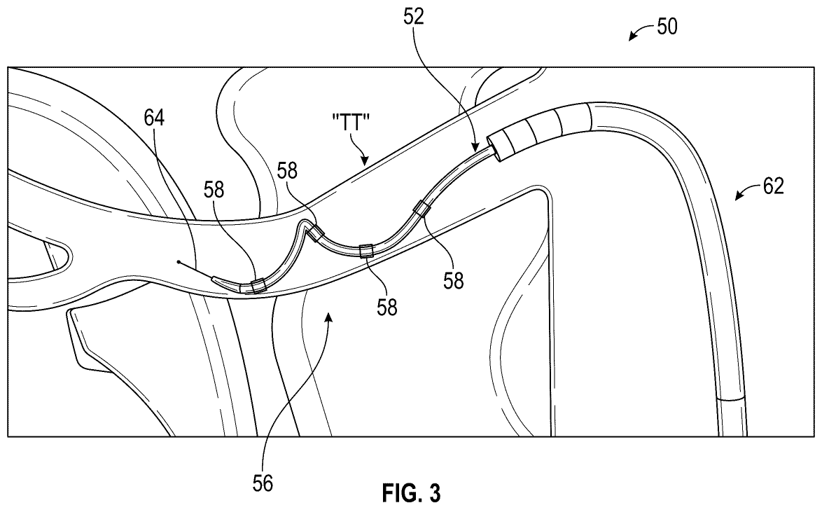

The therapy system 10 includes a workstation 20, a therapeutic device 50 operably coupled to the workstation 20, and in embodiments, an imaging device 14, which may be operably coupled to the workstation 20. The patient “P” is shown lying on an operating table 12 with the therapeutic device 50 inserted through a portion of the patient's femoral artery, although it is contemplated that the therapeutic device 50 may be inserted into any suitable portion of the patient's vascular network that is in fluid communication with a desired blood vessel for therapy (e.g., renal, hepatic, mesenteric, splanchnic, or other arteries enervated by the SNS). Although generally described as having one therapeutic device 50, it is envisioned that the therapy system 10 may employ any suitable number of therapeutic devices 50. The therapeutic devices 50 may employ the same or different modalities and may be operably coupled to the workstation 20. Further, the therapeutic device 50 may employ a guidewire 64 or a guide catheter 62 (FIG. 3) without departing from the scope of the present disclosure.

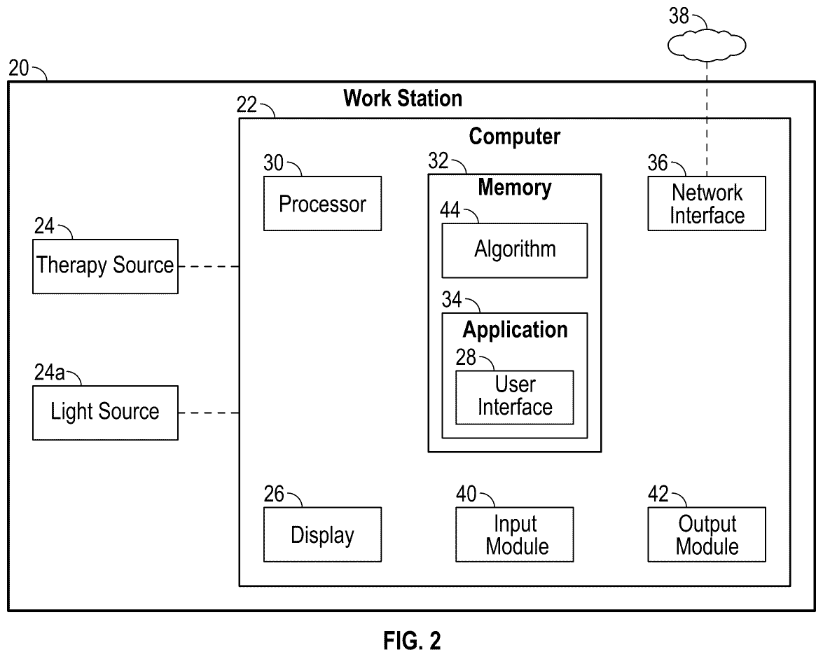

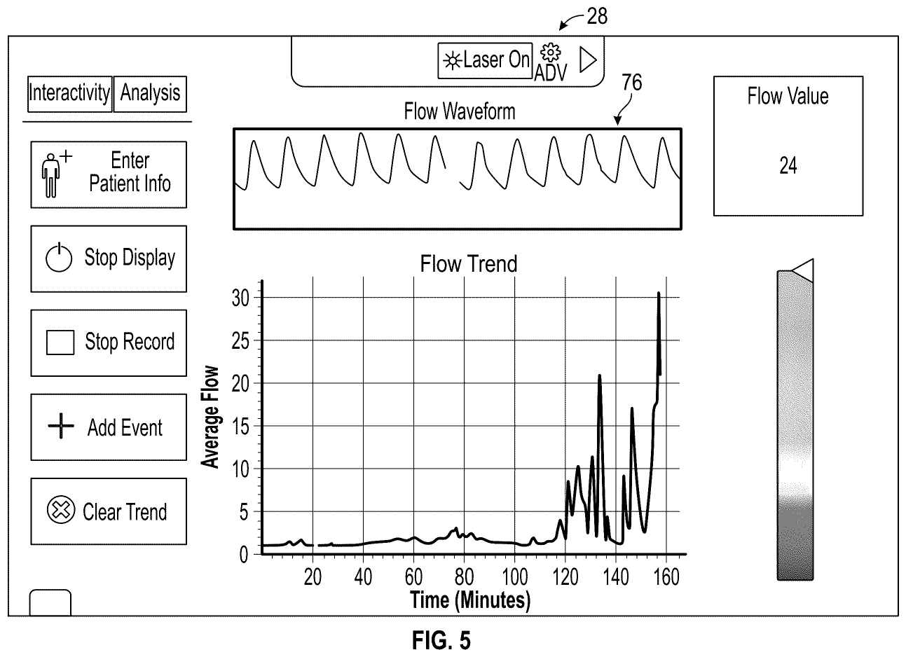

Continuing with FIG. 1 and with additional reference to FIG. 2, the workstation includes a computer 22 and a therapy source 24 (e.g., an RF generator, a microwave generator, an ultrasound generator, a cryogenic medium source, a chemical source, etc.) operably coupled to the computer 22. The computer 22 is coupled to a display 26 that is configured to display one or more user interfaces 28 (also illustrated in FIG. 5). The computer 22 may be a desktop computer or a tower configuration with display 26 or may include a laptop computer or other computing device. The computer 22 includes a processor 30 which executes software stored in a memory 32. The memory 32 may store one or more applications 34 and/or algorithms 44 to be executed by the processor 30. A network interface 36 enables the workstation 20 to communicate with a variety of other devices and systems via the Internet. The network interface 36 may connect the workstation 20 to the Internet via a wired or wireless connection. Additionally, or alternatively, the communication may be via an ad hoc Bluetooth® or wireless network enabling communication with a wide-area network (WAN) and/or a local area network (LAN). The network interface 36 may connect to the Internet via one or more gateways, routers, and network address translation (NAT) devices. The network interface 36 may communicate with a cloud storage system 38, in which further data, image data, or videos may be stored. The cloud storage system 38 may be remote from or on the premises of the hospital or clinic such as in a control or hospital information technology room. It is envisioned that the cloud storage system 38 could also serve as a host for more robust analysis of acquired images data or images (e.g., additional or reinforcement data for analysis and/or comparison, medical images (e.g., fluoroscopic, computed tomography (CT), magnetic resonance imaging (MRI), cone-beam computed tomography (CBCT), etc.) An input module 40 receives inputs from an input device such as a keyboard, a mouse, voice commands, amongst others. An output module 42 connects the processor 30 and the memory 32 to a variety of output devices such as the display 26. In embodiments, the display 26 may be a touchscreen display. The workstation 20 may also include a light source 24a, which is capable of generating one or more sources of light for transmission through a fiber-optic cable or other means to a diagnostic device 70 for use in the systems and methods described herein for quantification of sympathetic nerve activity or changes in sympathetic nerve activity. In embodiments, the light source 24a may also be included within the diagnostic device 70 itself.

FIG. 3 depicts an embodiment of a therapeutic device 50 in accordance with the disclosure. The therapeutic device 50 includes an elongated shaft 52 having a handle (not shown) disposed on a proximal end of the elongated shaft 52. The elongated shaft 52 of the therapeutic device 50 is configured to be advanced within a portion of the patient's vasculature, such as a femoral artery or other suitable portion of patient's vascular network that is in fluid communication with the patient's renal artery, or any other suitable target vessel. As depicted in FIG. 3, the elongated shaft 52 may be configured to be received within a portion of a guide catheter or guide sheath (such as a 6F guide catheter) 62 that is utilized to navigate the therapeutic device 50 to a desired location at which point the guide catheter 62 is retracted to uncover the therapeutic portion 56 of the therapeutic device 50 which in the embodiment shown includes a plurality of monopolar electrodes 58. The elongated shaft 52 of the therapeutic device 50 may further include an aperture (not shown) that is configured to slidably receive a guidewire 64 over which the therapeutic device 50, either alone or in combination with the guide catheter 62, are advanced. In this manner, the guidewire 64 is utilized to guide the therapeutic device 50 to the target tissue using over-the-wire (OTW) or rapid exchange (RX) techniques, at which point the guide wire may be partially or fully removed from the therapeutic device 50.

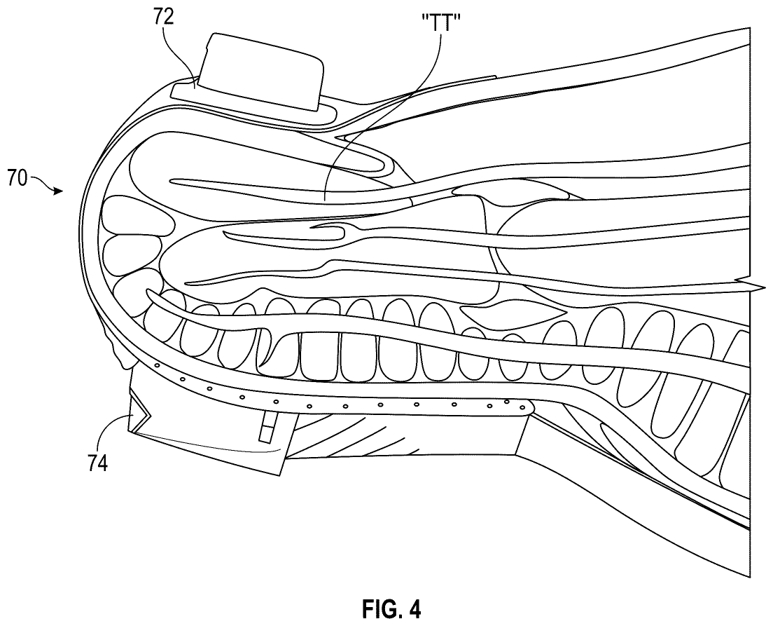

As can be appreciated, navigating the therapeutic device 50 within the patient's femoral artery, or other suitable vasculature, to assess whether a patient is a candidate for denervation therapy is an invasive procedure. The diagnostic systems and methods described herein allow for non-invasive assessment of the patient's physiological properties for candidacy for denervation therapy. FIG. 4 depicts an embodiment of a diagnostic device 70 in accordance with the disclosure. The diagnostic device 70 includes a light emitter 72 and a photodetector 74 for detecting light recovered from the light emitter 72 after interaction with target tissue. The light emitter 72 emits partially coherent or coherent light and may be a diode laser or a vertical-cavity surface-emitting laser (VCSEL) laser, amongst others. The light emitted by the light emitter 72 may be within the near-infrared spectrum, and in embodiments may include a wavelength between about 700 nm and about 900 nm. The light emitter 72 may be disposed in substantially direct contact with tissue of the patient or in embodiments, may be disposed in spaced relation to the tissue of the patient. It is envisioned that the light emitter 72 may be operably coupled to the light source 24a via a light transmitting conduit (e.g., fiber optic cable, etc.) operably coupled to the diagnostic device 70.

The photodetector 74 includes one or more light sensitive elements (not shown) and in embodiments, may be an image sensor. In embodiments, the photodetector 74 may be a silicon-based camera sensor, such as a CMOS or CCD image sensor, amongst others. As can be appreciated, the photodetector 74 detects light recovered from the light emitter 72 after interaction with the target tissue. It is contemplated that the photodetector 74 may generate one or more signals related to the detected light and transmit the generated signals to the computer 20. The signals generated by the photodetector 74 comprise quantifiable information about the intensity of light detected at one or more light sensitive elements at a point in time or over a course of time (including but not limited to spatial or temporal changes in intensity). In embodiments, the signals may include information about the absorption or scattering events within the tissue and may be analog or digital signals.

Continuing with FIG. 4, the diagnostic device 70 may be positioned in any suitable position or orientation relative to the target tissue such that the light emitter 72 and the photodetector 74 may be placed in contact or non-contact geometries or in reflectance or transmission geometries. In embodiments, the light emitter 72 and the photodetector 74 may be disposed adjacent to one another on a single side of the target tissue or may be disposed on opposing sides of the target tissue. In this manner, the diagnostic device 70 may be disposed on or coupled to a portion of the target tissue or may be free-standing or coupled to a structure that is independent from the target tissue. In embodiments, the diagnostic device may be a portable or wearable device, such as a watch, wristband, armband, ankle bracelet, belt, skin patches, ear-clips, finger clips, amongst others and may be remotely (e.g., wirelessly, etc.) or directly (e.g., wired, etc.) coupled to the workstation 20.

The light emitter 72 emits light towards the target tissue for a predetermined period of time, at least a portion of the light is scattered internally within the target tissue and detected by the photodetector 74. The predetermined amount of time during which the light is emitted from the light emitter 72 is long enough that the photodetector 74 can detect changes that occur within the target tissue. Changes in the target tissue during the emission of light alter the path of the emitted light or properties of the detected light. Dynamic properties of light scattering particles, such as rate of motion (e.g., flow rate) can be observed or measured and a peripheral blood flow waveform 76 can be generated and displayed on the user interface 28. The computer 20 may record the detected signals from the photodetector 74 and store the detected signals within the memory 32. The computer 20 analyzes the detected signals received from the photodetector 74 and determines information about the target tissue, such as a flow rate of blood flowing within the target tissue.

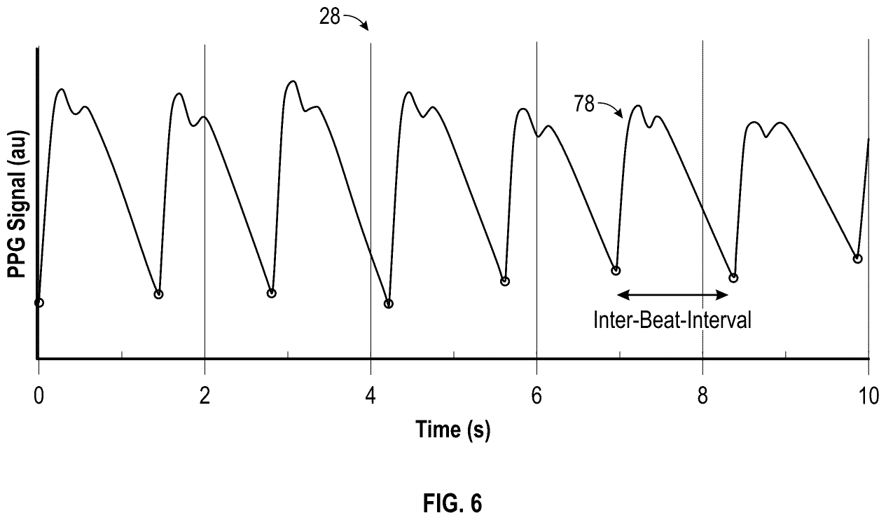

The photodetector 74 measures or detects changes in absorption of the light emitted from the light emitter 72. The computer 20 analyses the measurements from the photodetector 74 and generates a photoplethymogram (PPG), which can be displayed on the user interface 28 (FIG. 6). The PPG and blood flow signals closely align with arterial blood pressure and therefore, the PPG signals can form an arterial blood pressure waveform 78 over a period of time. Although generally described as using the same light emitter 72 and photodetector 74, it is envisioned that the flow rate measurements (e.g., peripheral blood flow waveform 76) and the PPG (e.g., arterial blood pressure waveform 78) may be generated using separate light emitters 72 and/or photodetectors 74 without departing from the scope of the present disclosure.

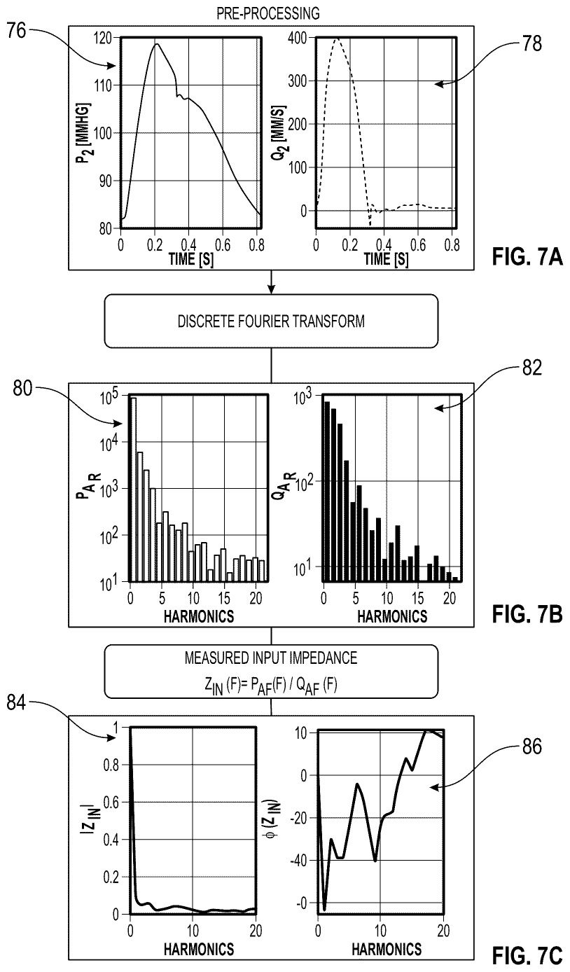

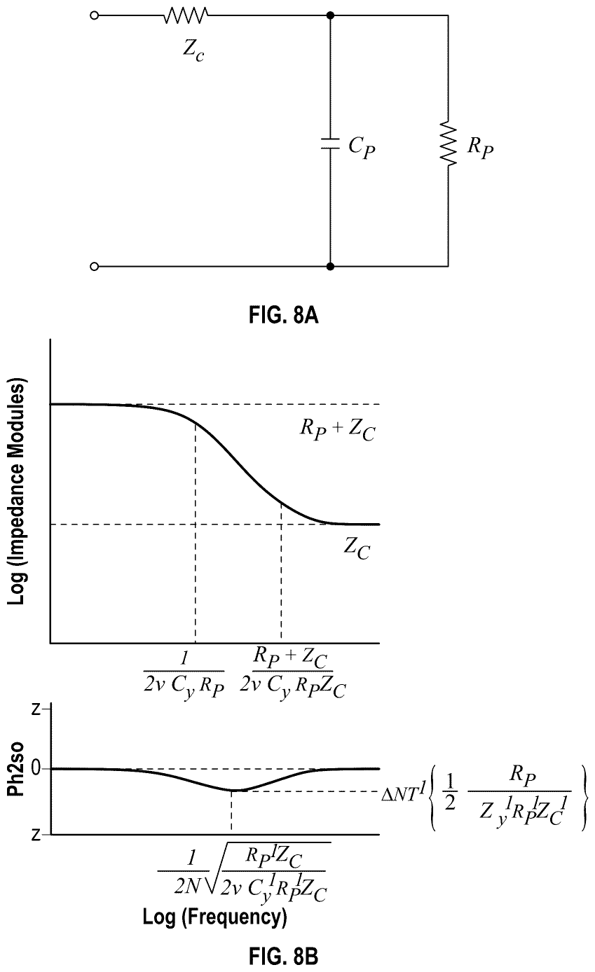

With reference to FIGS. 7A-C and 8, the computer 20 determines the peripheral blood flow waveform 76 and the arterial blood pressure waveform 78 over a period of time (FIG. 7A). Thereafter, the computer 20 calculates a Power Spectral Density (PSD) 80, 82 for each of the peripheral blood flow waveform 76 and the arterial blood pressure waveform 78 over the period of time (FIG. 7B). PSD is a measure of a signal's power content versus frequency and may be calculated in a frequency domain using the Fourier transform (FT), or fast Fourier transform (FFT), although it is contemplated that the PSD for each of the peripheral blood flow waveform 76 and/or the arterial blood pressure waveform 78 may be calculated using any suitable digital signal processing technique without departing from the scope of the present disclosure. With the PSD 80, 82 calculated for each of the peripheral blood flow waveform 76 and the arterial blood pressure waveform 78, the computer 20 conducts a power spectral analysis of arterial mechanical properties, such as peripheral arteriolar dimensions (e.g., resistance), aortic wall stiffness (e.g., compliance), wave reflections (e.g., reflectance), using an aortic input impedance spectrum 84, 86 (FIG. 7C). The aortic input impedance spectrum 84, 86 quantifies frequency independent and dependent components of left ventricular afterload, such as peripheral arterial resistance and arterial compliance. The computer 20 calculates the aortic input impedance spectrum 84, 86 using the quantified arterial blood flow waveform 76 and the arterial blood pressure waveform 78. As can be appreciated, the arterial mechanical properties can be derived from the magnitude or phase of the aortic input impedance spectrum 84, 86 using a mechanical or electrical model (e.g., an analogue model) of the arterial circulation (FIGS. 8A and 8B). In one non-limiting embodiment, the computer 22 interprets the aortic input impedance spectrum 84, 86 using the Windkessel model, such as the three-element Windkessel model, although it is contemplated that suitable model capable of interpreting the aortic input impedance spectrum 84, 86 may be utilized without departing from the scope of the present disclosure.

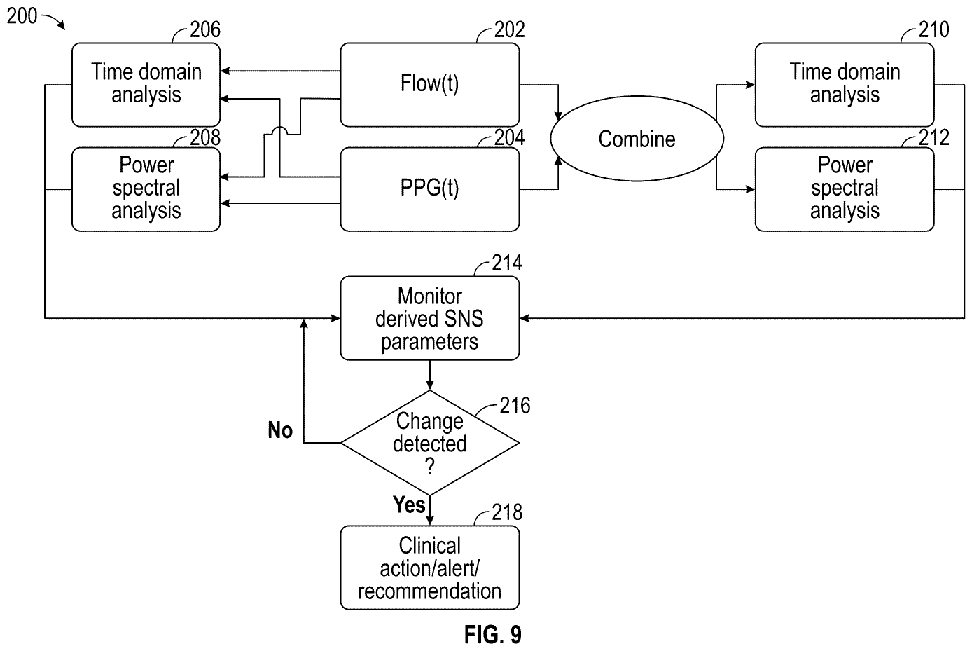

With reference to FIG. 9, a method of quantifying a relative level of global sympathetic nerve activity, as well as relative changes in activity, by analyzing components of the measured peripheral blood flow waveform 76 and the PPG signals (e.g., arterial blood pressure waveform 78) over a period of time alone, or in combination, in the time or frequency domain is illustrated and generally identified by reference numeral 200. The measured peripheral blood flow 202 and the PPG signal 204 can be analyzed in various combinations, such as individually in the time domain 206, individually in the frequency domain (PSD) 208, combined in the time domain 210 (e.g., the mean PPG signal amplitude and the mean measured peripheral blood flow amplitude), combined in the frequency domain (PSD) 212 (e.g., resistance, compliance, amplitude, phase, etc.), amongst others. If analyzed in combination, it is envisioned that within each combination, various features of the signal can be derived, including both time domain (e.g., mean, maximum, minimum, etc.) or frequency domain (PSD) parameters (e.g., magnitude, phase, etc.). Within the frequency domain 212, it is contemplated that the analysis may consist of low and high frequency bands, multiple bands, or any other frequency band of interest. In one non-limiting embodiment, the very low frequency band is analyzed, which is within the range of SNS firing (e.g., vasomotion).

Within each combination, the computer 20 monitors and derives parameters indicative of SNS activity in the basal steady state or following a perturbation of the SNS 214. It is envisioned that perturbations of the SNS can be intentionally applied in a clinical setting or could naturally occur in the case of long-term monitoring (e.g., over a single night, over several days and nights, etc.) of peripheral blood flow and PPG signals in a clinical setting or outside of a clinical setting (e.g., the patient's home, workplace, etc.).

For each of the tracked peripheral blood flow and PPG signals, a normal range or normal pattern of variation is defined as well as one or more thresholds for detecting a deviation from the defined normal range or pattern of variation, which can be stored in the memory 32 or be uploaded or otherwise set in a memory (not shown) associated with a portable device, such as a wristwatch, etc. It is envisioned that the defined normal range or normal pattern of variation may be expressed as either an absolute value or a proportional change without departing from the scope of the present disclosure. If no change is detected or if the tracked peripheral blood flow or PPG signals do not deviate from the defined normal range or pattern of variation or otherwise exceed/fall below the predetermined thresholds 216, the process returns to monitoring the tracked peripheral blood flow and PPG signals 214. If the tracked peripheral blood flow or PPG signals deviate from the defined normal range or pattern of variation or otherwise exceed/fall below the predetermined thresholds 216, the computer 20 may issue an alert (e.g., visual, auditory, haptic, etc.), display a clinical action associated with the deviation, and/or provide a recommendation regarding potential diagnostic decisions based upon the measured data 218.

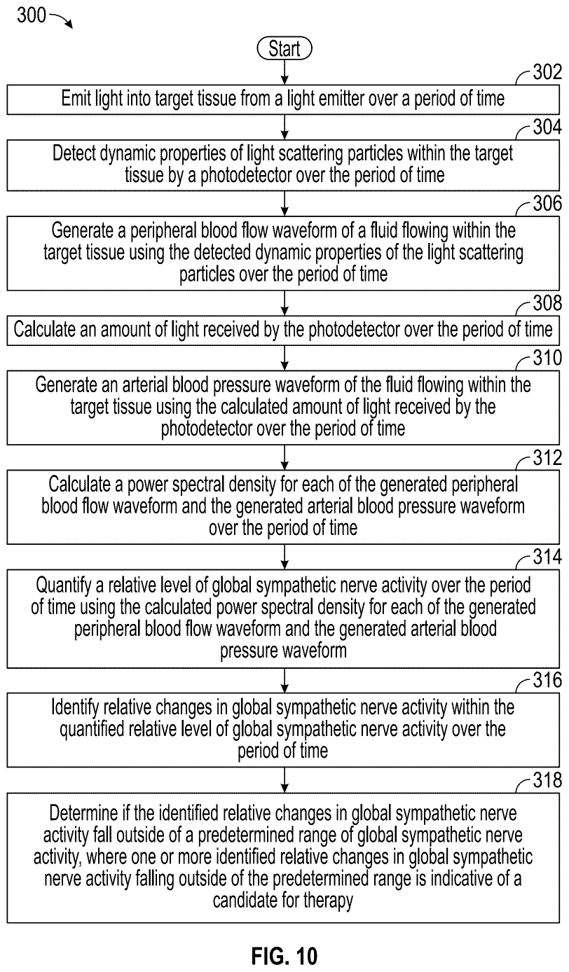

With reference to FIG. 10, a method of assessing a candidate for therapy is illustrated and generally identified by reference numeral 300. In step 302, light is emitted into target tissue from the light emitter 72 over a period of time. The photodetector 74 detects dynamic properties of light scattering particles within the target tissue over the period of time in step 304. In step 306, a peripheral blood flow waveform 76 of a fluid flowing within the target tissue is generated using the detected dynamic properties of the light scattering particles over the period of time. In step 308, an amount of light received by the photodetector 74 is measured over the period of time and in step 310, an arterial blood pressure waveform 78 of the fluid flowing within the target tissue is generated using the calculated amount of light received by the photodetector 74 over the period of time. In step 312, a power spectral density for each of the generated peripheral blood flow waveform 76 and the generated arterial blood pressure waveform 78 is calculated over the period of time. A relative level of global sympathetic nerve activity over the period of time is quantified in step 314 using the calculated power spectral density for each of the generated peripheral blood flow waveform and the generated arterial blood pressure waveform. In step 316, relative changes in global sympathetic nerve activity within the quantified relative level of global sympathetic nerve activity over the period of time is identified and in step 318, a determination is made regarding whether the identified relative changes in global sympathetic nerve activity fall outside of a predetermined range of global sympathetic nerve activity, where one or more identified relative changes in global sympathetic nerve activity falling outside of the predetermined rang is indicative of a candidate for therapy.

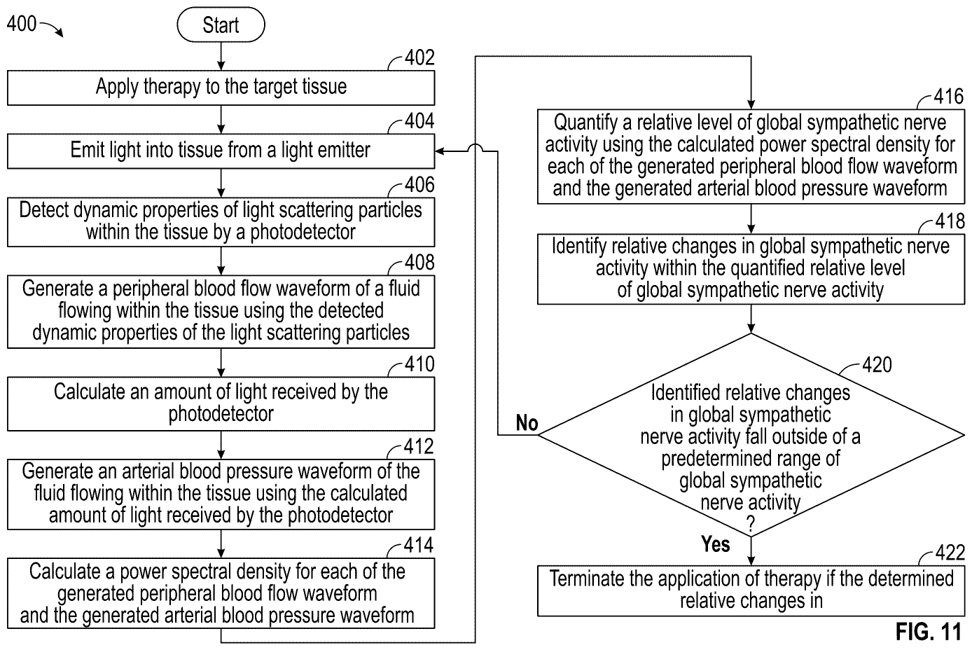

With reference to FIG. 11, a method of assessing and performing a therapeutic procedure is illustrated and generally identified by reference numeral 400. After navigation of a therapeutic device to target tissue, therapy is applied to the target tissue in step 402. During the application of therapy to the target tissue, light is emitted into tissue from a light emitter in step 404. In step 406, dynamic properties of light scattering particles within the tissue are detected by a photodetector and in step 408, a peripheral blood flow waveform of a fluid flowing within the tissue is generated using the detected dynamic properties of the light scattering particles. In step 410, an amount of light received by the photodetector is calculated, and in step 412, an arterial blood pressure waveform of the fluid flowing within the tissue is calculated using the calculated amount of light received by the photodetector. In step 414, a power spectral density for each of the generated peripheral blood flow waveform and the generated arterial blood pressure waveform is calculated. In step 416, a relative level of global sympathetic nerve activity is quantified using the calculated power spectral density for each of the generated peripheral blood flow waveform and the generated arterial blood pressure waveform. In step 418, relative changes in global sympathetic nerve activity within the quantified relative level of global sympathetic nerve activity is identified and in step 420, it is determined whether the identified relative changes in global sympathetic nerve activity falls outside of a predetermined range of global sympathetic nerve activity. If the identified relative changes in global sympathetic nerve activity does not fall outside the predetermined range of global sympathetic nerve activity, the method returns to step 404 to continue emitting light into the tissue. If the identified relative changes in global sympathetic nerve activity falls outside of the predetermined range of global sympathetic nerve activity, in step 422, the application of therapy to the target tissue is terminated.

Although FIG. 11 described a method wherein the change in SNS activity is employed to determine an end point for application of therapy, the disclosure is not so limited. In some embodiments, the method 300 may be undertaken to ensure that the patient is a candidate for denervation. Denervation to a desired blood vessel of the patient may be undertaken, and then method 300 may be undertaken a second time to assess the efficacy of the denervation. If sufficient change is detected (e.g., as in step 424) then the therapy is terminated. Otherwise, denervation can be continued for a time, or the parameters of the denervation (e.g., power, duration, location) may be altered and denervation again undertaken. This process may be repeated until a denervation has been achieved or therapy limits are reached without achieving the desired denervation.

Returning to FIG. 3, in embodiments where the therapeutic device 50 is an RF ablation catheter, the therapeutic portion 56 includes one or more electrodes 58 disposed on the shaft 52 that are configured to apply denervation therapy to the target tissue. It is envisioned that the one or more electrodes 58 may be disposed in spaced relation to each other and configured to contact the blood vessel walls suitable configuration, such as a helical, expanded configuration or by manipulating the therapeutic portion 56 to contact tissue walls (e.g., in the case of a linear arrangement). In one non-limiting embodiment, the therapeutic assembly 56 is configured to be transitioned from an initial, undeployed state having a generally linear profile, to a second, deployed or expanded configuration, where the therapeutic assembly 56 forms a generally spiral and/or helical configuration (FIG. 3) for delivering therapy at the treatment site and providing therapeutically-effective electrically and/or thermally induced renal neuromodulation. In this manner, when in the second, expanded configuration, the therapeutic assembly 56 is pressed against or otherwise contracts the walls of the patient's vasculature. Although generally described as transitioning to a spiral and/or helical configuration, it is envisioned that the therapeutic assembly 56 may be deployed to any suitable shape. In embodiments, the therapeutic assembly 56 may be capable of being placed in any suitable numbers of configurations depending upon the design needs of the therapeutic device 50 or the type of therapeutic procedure being performed. As shown herein, the therapeutic device 50 includes four electrodes 58. However, the present disclosure is not so limited and the therapeutic device 50 may have more or fewer electrodes 58 without departing from the scope of the present disclosure. One of skill in the art will recognize that the electrodes 58 may be replaced with ultrasound transducers, microwave antennae, ports for delivery of cryoablation medium or chemical medium and other implements and/or ablation and denervation modalities without departing from the scope of the present disclosure

As illustrated in the figures, the electrodes 58 are disposed in spaced relation to one another along a length of the therapeutic device 50 forming the therapeutic portion 56. As will be appreciated, these electrodes 58 are in communication with the therapy source 24 which produces, for example, monopolar RF energy to denervate the sympathetic nerves of the relevant blood vessel. Additionally, or alternatively, the electrodes 58 may deliver RF energy independently of one another (e.g., monopolar), simultaneously, selectively, sequentially, and/or between any desired combination of the electrodes 58 (e.g., bipolar).

In one embodiment, the therapeutic device 50 may be a cryotherapy device where the therapeutic portion 56 may include one therapy delivery element, such as an occlusive balloon, a non-occlusive balloon, or other balloon permitting the flow of blood, etc. In this embodiment, the therapy source 24 may include a cryogen or coolant source or means to generate a cryogen. It is envisioned that the therapeutic device 50 may be a microwave energy device where the therapeutic portion 56 may include one or more therapy delivery elements, such as a microwave antenna. In this embodiment, the therapy source 24 may be a microwave energy generator that is operably coupled to the microwave antenna. It is contemplated that the therapeutic device may be an ultrasound device where the therapeutic portion 56 may include one or more therapy delivery elements, such as an ultrasound transducer, etc. In this embodiment, the therapy source 24 may be a radio-frequency energy generator or the like that is operably coupled to the ultrasound transducer. In embodiments, the therapeutic device 50 may be a chemical denervation device where the therapeutic portion 56 may include one or more cannulas or needles for the administration of a chemical denervation agent. In this embodiment, the therapy source 24 may be a chemical denervation agent source that is operably coupled to the therapeutic portion 56. Those having skill in the art will recognize that the therapeutic device 50, the therapeutic portion 56, and the therapy source 24 may be any suitable combination of devices capable of performing a denervation procedure.

Although described generally hereinabove, it is envisioned that the memory 32 may include any non-transitory computer-readable storage media for storing data and/or software including instructions that are executable by the processor 30 and which control the operation of the workstation 20 and, in some embodiments, may also control the operation of the therapeutic device 50, and/or imaging device 70. In an embodiment, memory 32 may include one or more storage devices such as solid-state storage devices, e.g., flash memory chips. Alternatively, or in addition to the one or more solid-state storage devices, the memory 32 may include one or more mass storage devices connected to the processor 30 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 30. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the therapy source 24.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

The following examples are a non-limiting list of clauses in accordance with one or more techniques of this disclosure.
Example 1. A system for performing a diagnostic procedure, comprising: a diagnostic device, the diagnostic configured for placement proximate tissue, wherein a light emitter and a photodetector are disposed on the diagnostic device; a computing device including a processor and a memory storing instructions, which when executed by the processor, cause the computing device to: deliver light from the light emitter into the tissue for a period of time; detect dynamic properties of light scattering particles within the tissue using the photodetector over the period of time; generate a peripheral blood flow waveform of a fluid flowing within the tissue using the detected dynamic properties of the light scattering particles over the period of time; calculate a power spectral density for the generated peripheral blood flow waveform over the period of time; quantify a relative level of global sympathetic nerve activity over the period of time using the calculated power spectral density for the generated peripheral blood flow waveform; identify relative changes in global sympathetic nerve activity within the quantified relative level of global sympathetic nerve activity over the period of time; and determine if the identified relative changes in global sympathetic nerve activity fall outside of a predetermined range of global sympathetic nerve activity, wherein one or more identified relative changes in global sympathetic nerve activity falling outside of the predetermined range is indicative of a candidate for therapy.
Example 2. The system according to Example 1, wherein the instructions, when executed by the processor, cause the computing device to calculate the power spectral density the generated peripheral blood flow waveform over the period of time using the fast Fourier transform.
Example 3. The system according to Example 2, wherein calculating the power spectral density includes calculating the power spectral density for the generated peripheral blood flow waveform over the period of time using the fast Fourier transform.
Example 4. The system according to Example 1, wherein the instructions, when executed by the processor, cause the computing device to calculate an aortic input impedance spectrum for the generated peripheral blood flow waveform over the period of time using the calculated power spectral density for the generated peripheral blood flow waveform.
Example 5. The system according to Example 4, wherein the instructions, when executed by the processor, cause the computing device to quantify the relative level of global sympathetic nerve activity over the period of time using one of the calculated power spectral density or the calculated aortic impedance spectrum.
Example 6. The system according to Example 4, wherein the instructions, when executed by the processor, cause the computing device to calculate the aortic input impedance spectrum for the generated peripheral blood flow waveform over the period of time using a Windkessel model.
Example 7. The system according to Example 5, wherein the instructions, when executed by the processor, cause the computing device to calculate the aortic input impedance spectrum for the generated peripheral blood flow waveform over the period of time using the three-element Windkessel model.
Example 8. The system according to Example 5, wherein the instructions, when executed by the processor, cause the computing device to calculate an amount of light received by the photodetector over the period of time.
Example 9. The system according to Example 8, wherein the instructions, when executed by the processor, cause the computing device to generate an arterial blood pressure waveform of the fluid flowing within the target tissue using the calculated amount of light received by the photodetector over the period of time.
Example 10. The system according to Example 9, wherein the instructions, when executed by the processor, cause the computing device to calculate the aortic input impedance spectrum of a combined generated peripheral blood flow waveform and generated arterial blood pressure waveform.
Example 11. The system according to Example 10, wherein the instructions, when executed by the processor, cause the computing device to combine the generated peripheral blood flow waveform and the generated arterial blood pressure waveform using a derived one or more of a determined resistance, compliance, amplitude, or phase from the generated peripheral blood flow waveform and the generated arterial blood pressure waveform.
Example 12. The system according to Example 9, wherein the instructions, when executed by the processor, cause the computing device to calculate a power spectral density of a combined generated peripheral blood flow waveform and generated arterial blood pressure waveform.
Example 13. The system according to Example 12, wherein the instructions, when executed by the processor, cause the computing device to combine the generated peripheral blood flow waveform and the generated arterial blood pressure waveform using a derived mean arterial blood pressure amplitude and a derived mean peripheral blood flow waveform from the generated peripheral blood flow waveform and the generated arterial blood pressure waveform over the period of time.
Example 14. The system according to Example 1, further comprising intentionally applying a sympathetic stimulus to the candidate for therapy, wherein identifying relative changes in global sympathetic nerve activity includes identifying relative changes in global sympathetic nerve activity during the intentionally applied sympathetic stimulus.
Example 15. The system according to Example 14, wherein the intentionally applied sympathetic stimulus is selected from the group consisting of a cold pressor, a hand rip exercise, mental stress, a Valsalva or Mueller maneuver, an acute catécholamine injection, orthostasis, and variable rate pacing.
Example 16. The system according to Example 1, wherein the instructions, when executed by the processor, cause the computing device to identify relative changes in global sympathetic nerve activity due to naturally occurring sympathetic stimulus.
Example 17. The system according to Example 16, wherein the naturally occurring sympathetic stimulus is selected from the group consisting of circadian variation, sleep apnea breathing, orthostasis, intense physical activity, and respiration.
Example 18. The system according to Example 1, wherein the diagnostic device is a portable, wearable device, the portable, wearable device wirelessly couplable to the computing device.
Example 19. The system according to Example 1, wherein the diagnostic device is operably coupled to the computing device using a wired connection.
Example 20. The system according to Example 1, wherein the instructions, when executed by the processor, cause the computing device to issue one or more of a clinical action, an alert, or a recommendation based upon whether the identified relative changes in global sympathetic nerve activity fall outside of the predetermined range.
Example 21. A method of assessing a candidate for therapy, comprising: causing, by a computing device, a light emitter to emit light into target tissue of a patient over a period of time; receiving, by the computing device, from a photodetector, a signal indicative of dynamic properties of light scattering particles within the target tissue over the period of time; generating, by the computing device, a peripheral blood flow waveform of a fluid flowing within the target tissue using the dynamic properties of the light scattering particles over the period of time; calculating, by the computing device, a power spectral density for the generated peripheral blood flow waveform the period of time; quantifying, by the computing device, a relative level of global sympathetic nerve activity over the period of time using the calculated power spectral density for the generated peripheral blood flow waveform; identifying, by the computing device, relative changes in global sympathetic nerve activity within the quantified relative level of global sympathetic nerve activity over the period of time; and determining, by the computing device, if the identified relative changes in global sympathetic nerve activity fall outside of a predetermined range of global sympathetic nerve activity, wherein one or more identified relative changes in global sympathetic nerve activity falling outside of the predetermined range is indicative of the patient being a candidate for therapy.
Example 22. The method according to Example 21, wherein calculating the power spectral density includes calculating the power spectral density for the generated peripheral blood flow waveform over the period of time using a Fourier transform.
Example 23. The method according to Example 22, wherein calculating the power spectral density includes calculating the power spectral density for the generated peripheral blood flow waveform over the period of time using the fast Fourier transform.
Example 24. The method according to Example 21, further comprising calculating, by the computing device, an aortic input impedance spectrum for the generated peripheral blood flow waveform over the period of time using the calculated power spectral density for the generated peripheral blood flow waveform.
Example 25. The method according to Example 24, wherein quantifying the relative level of global sympathetic nerve activity includes quantifying the relative level of global sympathetic nerve activity over the period of time using one of the calculated power spectral density or the calculated aortic impedance spectrum.
Example 26. The method according to Example 24, wherein calculating the aortic input impedance spectrum includes calculating the aortic input impedance spectrum for the generated peripheral blood flow waveform over the period of time using a Windkessel model.
Example 27. The method according to Example 25, wherein calculating the aortic input impedance spectrum includes calculating the aortic input impedance spectrum for the generated peripheral blood flow waveform over the period of time using the three-element Windkessel model.
Example 28. The method according to Example 25, further comprising calculating, by the computing device, an amount of light received by the photodetector over the period of time.
Example 29. The method according to Example 28, further comprising generating, by the computing device, an arterial blood pressure waveform of the fluid flowing within the target tissue using the calculated amount of light received by the photodetector over the period of time
Example 30. The method according to Example 29, wherein calculating the aortic input impedance spectrum includes calculating the aortic input impedance spectrum of a combined generated peripheral blood flow waveform and generated arterial blood pressure waveform.
Example 31. The method according to Example 30, wherein combining the generated peripheral blood flow waveform and the generated arterial blood pressure waveform includes deriving one or more of a determined resistance, compliance, amplitude, or phase from the generated peripheral blood flow waveform and the generated arterial blood pressure waveform.
Example 32. The method according to Example 29, wherein calculating the power spectral density includes calculating a power spectral density of a combined generated peripheral blood flow waveform and generated arterial blood pressure waveform.
Example 33. The method according to Example 32, wherein combining the generated peripheral blood flow waveform and the generated arterial blood pressure waveform includes deriving a mean arterial blood pressure amplitude and a mean peripheral blood flow waveform amplitude from the generated peripheral blood flow waveform and the generated arterial blood pressure waveform over the period of time.
Example 34. The method according to Example 21, further comprising intentionally applying a sympathetic stimulus to the candidate for therapy, wherein identifying relative changes in global sympathetic nerve activity includes identifying relative changes in global sympathetic nerve activity during the intentionally applied sympathetic stimulus.
Example 35. The method according to Example 34, wherein the intentionally applied sympathetic stimulus is selected from the group consisting of a cold pressor, a hand rip exercise, mental stress, a Valsalva or Mueller maneuver, an acute catecholamine injection, orthostasis, and variable rate pacing.
Example 36. The method according to Example 21, wherein identifying relative changes in global sympathetic nerve activity includes identifying relative changes in global sympathetic nerve activity due to naturally occurring sympathetic stimulus.
Example 37. The method according to Example 36, wherein the naturally occurring sympathetic stimulus is selected from the group consisting of circadian variation, sleep apnea breathing, orthostasis, intense physical activity, and respiration.
Example 38. The method according to Example 21, further comprising issuing one or more of a clinical action, an alert, or a recommendation based upon whether the identified relative changes in global sympathetic nerve activity fall outside of the predetermined range.
Example 39. A method of assessing and performing a therapeutic procedure, comprising: navigating a therapeutic device to target tissue, the therapeutic device configured to apply therapy to the target tissue; applying therapy, by the therapeutic device, to the target tissue;
during the application of therapy to the target tissue, monitoring, by a computing device, global sympathetic nerve activity, wherein monitoring global sympathetic nerve activity includes: emitting light, from a light emitter, into tissue over a period of time; detecting, by the computing device, dynamic properties of light scattering particles within the tissue by a photodetector over the period of time; generating, by the computing device, a peripheral blood flow waveform of a fluid flowing within the tissue using the detected dynamic properties of the light scattering particles over the period of time; calculating, by the computing device, a power spectral density for the generated peripheral blood flow waveform over the period of time; quantifying, by the computing device, a relative level of global sympathetic nerve activity using the calculated power spectral density for the generated peripheral blood flow waveform; identifying, by the computing device, relative changes in global sympathetic nerve activity within the quantified relative level of global sympathetic nerve activity over the period of time; and determining, by the computing device, if the identified relative changes in global sympathetic nerve activity falls outside of a predetermined range of global sympathetic nerve activity; and terminating the application of therapy if the determined relative changes in global sympathetic nerve activity fall outside of the predetermined range.
Example 40. The method according to Example 39, further comprising adjusting, by the computing device, the applied therapy in response to the monitored global sympathetic nerve activity.
Example 41. The method according to Example 39, further comprising, after the application of therapy to the target tissue, monitoring, by the computing device, global sympathetic nerve activity a second time to determine, by the computing device, an efficacy of the application of therapy to the target tissue.
Example 42. The method according to Example 39, wherein calculating a power spectral density includes calculating the power spectral density for the generated peripheral blood flow waveform over the period of time using a Fourier transform.
Example 43. The method according to Example 42, wherein calculating the power spectral density includes calculating the power spectral density for the generated peripheral blood flow waveform over the period of time using the fast Fourier transform.
Example 44. The method according to Example 39, further comprising calculating, by the computing device, an aortic input impedance spectrum for the generated peripheral blood flow waveform over the period of time using the calculated power spectral density for the generated peripheral blood flow waveform.
Example 45. The method according to Example 44, wherein quantifying the relative level of global sympathetic nerve activity includes quantifying the relative level of global sympathetic nerve activity over the period of time using one of the calculated power spectral density or the calculated aortic input impedance spectrum.
Example 46. The method according to Example 44, wherein calculating the aortic input impedance spectrum includes calculating the aortic input impedance spectrum for the generated peripheral blood flow waveform over the period of time using a Windkessel model.
Example 47. The method according to Example 46, wherein calculating the aortic input impedance spectrum includes calculating the aortic input impedance spectrum for the generated peripheral blood flow waveform over the period of time using the three-element Windkessel model.
Example 48. The method according to Example 46, further comprising calculating, by the computing device, an amount of light received by the photodetector over the period of time.
Example 49. The method according to Example 48, further comprising generating, by the computing device, an arterial blood pressure waveform of the fluid flowing within the target tissue using the calculated amount of light received by the photodetector over the period of time.
Example 50. The method according to Example 49, wherein calculating the aortic input impedance spectrum includes calculating the aortic input impedance spectrum of a combined generated peripheral blood flow waveform and generated arterial blood pressure waveform.
Example 51. The method according to Example 50, wherein combining the generated peripheral blood flow waveform and the generated arterial blood pressure waveform includes deriving one or more of a determined resistance, compliance, amplitude, or phase from the generated peripheral blood flow waveform and the generated arterial blood pressure waveform.
Example 52. The method according to Example 39, wherein calculating the power spectral density includes calculating a power spectral density of a combined generated peripheral blood flow waveform and generated arterial blood pressure waveform.
Example 53. The method according to Example 52, wherein combining the generated peripheral blood flow waveform and the generated arterial blood pressure waveform includes deriving a mean arterial blood pressure amplitude and a mean peripheral blood flow waveform from the generated peripheral blood flow waveform and the generated arterial blood pressure waveform over the period of time.
Example 54. The method according to Example 39, wherein emitting light into tissue from a light emitter and detecting dynamic properties of light scattering particles within the tissue by a photodetector includes the light emitter and the photodetector being disposed on a diagnostic device.
Example 55. The method according to Example 54, wherein the diagnostic device is a portable, wearable device.
Example 56. The method according to Example 54, wherein the diagnostic device is disposed on a digit of a patient.

1. A system for performing a diagnostic procedure, comprising:
a diagnostic device, the diagnostic configured for placement proximate tissue, wherein a light emitter and a photodetector are disposed on the diagnostic device;
a computing device including a processor and a memory storing instructions, which when executed by the processor, cause the computing device to:
deliver light from the light emitter into the tissue for a period of time;
detect dynamic properties of light scattering particles within the tissue using the photodetector over the period of time;
generate a peripheral blood flow waveform of a fluid flowing within the tissue using the detected dynamic properties of the light scattering particles over the period of time;
calculate a power spectral density for the generated peripheral blood flow waveform over the period of time;
quantify a relative level of global sympathetic nerve activity over the period of time using the calculated power spectral density for the generated peripheral blood flow waveform;
identify relative changes in global sympathetic nerve activity within the quantified relative level of global sympathetic nerve activity over the period of time; and
determine if the identified relative changes in global sympathetic nerve activity fall outside of a predetermined range of global sympathetic nerve activity, wherein one or more identified relative changes in global sympathetic nerve activity falling outside of the predetermined range is indicative of a candidate for therapy.

2. The system according to claim 1, wherein the instructions, when executed by the processor, cause the computing device to calculate the power spectral density for the generated peripheral blood flow waveform over the period of time using a fast Fourier transform.

3. The system according to claim 1, wherein the instructions, when executed by the processor, cause the computing device to calculate an aortic input impedance spectrum for the generated peripheral blood flow waveform over the period of time using the calculated power spectral density for the generated peripheral blood flow waveform.

4. The system according to claim 3, wherein the instructions, when executed by the processor, cause the computing device to quantify the relative level of global sympathetic nerve activity over the period of time using one of the calculated power spectral density or the calculated aortic impedance spectrum.

5. The system according to claim 3 wherein the instructions, when executed by the processor, cause the computing device to calculate the aortic input impedance spectrum for the generated peripheral blood flow waveform over the period of time using a Windkessel model.

6. The system according to claim 4, wherein the instructions, when executed by the processor, cause the computing device to calculate an amount of light received by the photodetector over the period of time.

7. The system according to claim 6, wherein the instructions, when executed by the processor, cause the computing device to generate an arterial blood pressure waveform of the fluid flowing within the target tissue using the calculated amount of light received by the photodetector over the period of time.

8. The system according to claim 7, wherein the instructions, when executed by the processor, cause the computing device to calculate the aortic input impedance spectrum of a combined generated peripheral blood flow waveform and generated arterial blood pressure waveform.

9. The system according to claim 8, wherein the instructions, when executed by the processor, cause the computing device to combine the generated peripheral blood flow waveform and the generated arterial blood pressure waveform using a derived one or more of a determined resistance, compliance, amplitude, or phase from the generated peripheral blood flow waveform and the generated arterial blood pressure waveform.

10. The system according to claim 7, wherein the instructions, when executed by the processor, cause the computing device to calculate a power spectral density of a combined generated peripheral blood flow waveform and generated arterial blood pressure waveform.

11. The system according to claim 10, wherein the instructions, when executed by the processor, cause the computing device to combine the generated peripheral blood flow waveform and the generated arterial blood pressure waveform using a derived mean arterial blood pressure amplitude and a derived mean peripheral blood flow waveform from the generated peripheral blood flow waveform and the generated arterial blood pressure waveform over the period of time.

12. The system according to claim further comprising intentionally applying a sympathetic stimulus to the candidate for therapy, wherein identifying relative changes in global sympathetic nerve activity includes identifying relative changes in global sympathetic nerve activity during the intentionally applied sympathetic stimulus.

13. The system according to claim 12, wherein the intentionally applied sympathetic stimulus is selected from the group consisting of a cold pressor, a hand rip exercise, mental stress, a Valsalva or Mueller maneuver, an acute catecholamine injection, orthostasis, and variable rate pacing.

14. The system according to claim wherein the instructions, when executed by the processor, cause the computing device to identify relative changes in global sympathetic nerve activity due to a naturally occurring sympathetic stimulus.

15. The system according to claim 14, wherein the naturally occurring sympathetic stimulus is selected from the group consisting of circadian variation, sleep apnea breathing, orthostasis, intense physical activity, and respiration.

16. The system according to claim 1 wherein the diagnostic device is a portable, wearable device, the portable, wearable device wirelessly couplable to the computing device.

17. The system according to claim 1 wherein the instructions, when executed by the processor, cause the computing device to issue one or more of a clinical action, an alert, or a recommendation based upon whether the identified relative changes in global sympathetic nerve activity fall outside of the predetermined range.

18. The system according to claim 2, wherein calculating the power spectral density includes calculating the power spectral density for the generated peripheral blood flow waveform over the period of time using the fast Fourier transform.

19. The system according to claim 4, wherein the instructions, when executed by the processor, cause the computing device to calculate the aortic input impedance spectrum for the generated peripheral blood flow waveform over the period of time using the three-element Windkessel model.

20. The system according to claim 1, wherein the diagnostic device is operably coupled to the computing device using a wired connection.
